(19) United States
(12) Patent Application Publication
(10) Pub. No.: US 2025/0226096 A1
(43) Pub. Date: Jul. 10, 2025

(54) MACHINE LEARNING ARCHITECTURE FOR IMPROVED WELLNESS MONITORING

(71) Applicant: Abbott Diabetes Care Inc., Alameda, CA (US)

(72) Inventors: Rachelle Anne Kresch FISCHER, San Francisco, CA (US); Ravi Sankar KROTHAPALLI, Saratoga Springs, UT (US); Karthik NAKKEERAN, San Jose, CA (US); Ranaji KRISHNA, Berkeley, CA (US); Caroline Campbell KRYDER, San Francisco, CA (US); Amy Lynn MCKENZIE, Windach, Bayern (DE)

(21) Appl. No.: 19/009,550

(22) Filed: Jan. 3, 2025

Related U.S. Application Data
(60) Provisional application No. 63/617,722, filed on Jan. 4, 2024.

(51) Int. Cl. G16H 50/20; G06N 20/00

(52) U.S. Cl. CPC G16H 50/20; G06N 20/00

(57) ABSTRACT

Disclosed herein are system, method, and computer program product embodiments for a machine learning architecture for real-time medical data analysis and wellness event prediction. The architecture incorporates a trained machine learning model to predict wellness event sequences for users and various corrective features to ensure accuracy of the predicted sequences. Real-time analyte data is inputted into the machine learning model, generating an initial sequence of wellness events. False positives, false negatives, and missed events in the sequence may be identified and used to modify the initial sequence of wellness events. The modified sequence of wellness events may be presented via a graphical user interface along with user interface elements corresponding to the wellness events.

### CROSS-REFERENCE TO RELATED APPLICATIONS

This application claims priority to U.S. Provisional Application 63/617,722, filed on Jan. 4, 2024, which is incorporated by reference herein in its entirety.

### FIELD OF THE INVENTION

Embodiments of the present invention generally relate to methods and systems for increased accuracy and efficiency in predicting wellness events using analyte information. Additionally, embodiments of the present invention relate to methods and systems for generating personalized visual interface elements based on the improved predictions.

### BACKGROUND

The monitoring and management of wellness and nutrition in individuals can significantly benefit those at risk of or currently experiencing chronic health problems and those motivated to improve general wellness. Monitoring of wellness in individuals can be challenging because accurate monitoring is dependent on health data of the individual and reliable processing of that health data. Both collection of health data and reliable processing of the health data presents challenges in terms of how often the health data can be collected, the accuracy of the health data being collected, and the tools that are used to generate accurate visualizations and predictions based on that health data.

### SUMMARY

Accordingly, what is needed is a system that continuously monitors a patient's health data, including the patient's analyte information and processes the health data using a trained machine learning architecture to provide more accurate processing of the health data and for generating visualizations and recommendations based on that health data. As a non-limiting example, the machine learning architecture may be implemented within a continuous analyte monitoring system, where the use of a continuous analyte sensor provides real-time time analyte information and the use of trained machine learning models to detect health conditions and to generate predictions based on the real-time analyte information.

Conventional glucose detection systems frequently face significant challenges when attempting to accurately identify critical patterns within real-time glucose data. These challenges lead to potential delays in treatment decisions or to suboptimal management of diabetic conditions. The inherent complexity of glucose fluctuations—caused by factors such as meal intake, physical activity, stress, and circadian rhythms—combined with ineffectiveness of traditional systems to analyze these factors in real time, results in missed opportunities to recognize and respond to important trends. Moreover, conventional systems tend to rely on fixed threshold levels for detecting hyperglycemia or hypoglycemia, which lack the capability to detect more subtle trends or inflection points that could signal a pending health risk.

This disclosure addresses these deficiencies by introducing an improved machine learning model, guardrail component, and retrospective cleanup component within a system architecture specifically trained for real-time glucose data analysis with enhanced accuracy and responsiveness. In some aspects, the model is trained using labeled glucose datasets where each data point is annotated to identify specific patterns, such as the onset of a spike, a temporary dip, gradual declines, and other significant glucose trends. These patterns are inherently complex and are influenced by dynamic physiological and behavioral factors, making them difficult to capture using conventional approaches.

One technical improvement of this machine learning model and architecture include increases accuracy and confidence in differentiating between critical glucose events such as recognized starts and peaks and temporary or false events, such as intra-spike peaks and dips. Events can be identified at the closest visual local minima. This precision enhances the accuracy of spike detection, reducing false positives and negatives. The improved accuracy also reduces (but not necessarily eliminates) reliance on secondary retrospective algorithms to capture missed spikes. This streamlines the detection process and improves overall efficiency for predicting wellness events. One aspect of the architecture that results in this improvement is the interaction between the trained machine learning model, guardrail component, and retrospective cleanup component. The improved pattern detection accuracy also is provided via advanced training techniques on labeled data, which improves the precision with which crucial patterns—such as glucose trends that may indicate a pending spike—are detected in real time. The improved machine learning model is trained to account for various parameters within glucose data such as the glucose rate of change and identification of temporary peaks or dips that may otherwise be misclassified. By recognizing these complex interactions, the model enhances the accuracy and reliability of glucose detection beyond what fixed-threshold algorithms or basic statistical models could achieve.

Another technical improvement is enhanced visualization and user interfaces hat visually represents identified glucose trends with improved clarity and contextual insight. For example, the enhanced visualization may include automated visual markers for spikes, dips, and stable periods, along with trend forecasts. These visual cues enable more informed recommendations and insights—such as altering dietary habits, altering meal sequencing, recommended physical activity, or adjusting insulin dosing—based on a real-time understanding of how their glucose levels are changing. The enhanced visualizations represent improved responses to detected trends with enhanced real-time adjustments that reduce the time lag inherent in conventional glucose detection systems.

Another technical improvement is to the treatment recommendations and insights for providing more precise and timely therapy adjustments. Accurately identifying specific trends in user glucose levels ensures that the architecture generates appropriate responses—such as insulin administration or carbohydrate intake—at optimal times. This results in improved overall management of glucose levels, reducing the risk of hyperglycemia and hypoglycemia and ultimately leading to better patient health outcomes. Importantly, the ability to differentiate between a true spike peaks and ends and intra-spike peaks and dips ensures that corrective actions are taken only when appropriate, avoiding unnecessary interventions and minimizing the risk of hypoglycemia.

Another technical improvement is that is solves the cold start problem that is prevalent in current methods, which can require a waiting period of days, to establish a user baseline that determines spike start and endpoints. The machine learning model of the present disclosure is capable of generating predictions in less time (e.g., from days to only a few hours) by utilizing less data points to make predictions. This is possible because the datasets provided for training the model are based on annotations that meet clinician standards for spike starts and ends. A conventional rules-based approach needs days of historical data to establish a user baseline for identifying spike ends. The disclosed machine learning model adapts to the user baseline as it is trained on diverse user data, which enables it to begin generating predictions more quickly.

These improvements are not merely a mathematical algorithm but a technological enhancement that optimizes the way glucose detection systems function.

Accordingly, the disclosed machine learning model and architecture offers a technological solution to the technical problem of accurately detecting complex glucose patterns in real time. The improvement lies in enhancing the functioning of conventional glucose detection systems through pattern recognition techniques that leverage labeled datasets and interaction between the machine learning model and other guardrail components within the system architecture.

According to some embodiments, a machine learning architecture is implemented with a continuous analyte monitoring (CAM) system. The CAM system may be configured with one or more analyte sensors in communication with one or more reader devices and user devices, which are configured to receive the continuously monitored analyte information generated by the one or more analyte sensors. The machine learning architecture may include a machine learning model for generating predictions for wellness events related to a patient. The model may compile a wellness event sequence that includes one or more of the predicted wellness events that is displayed as part of a personalized visualization where each of the predicted wellness events may be separate user interface elements in the visualization.

The model may further include a guardrail component that is configured to receive the predicted wellness event sequence from the machine learning model. The guardrail component may also be configured to identity a false positive event or a false negative event in the predicted wellness event sequence based on an analysis of time-ordered events within the predicted wellness event sequence. The guardrail component may also further be configured to modify the predicted wellness event sequence based on the at least one of the false positive event or the false negative event. The machine learning architecture may be implemented as a device in the CAM system. The device may include a display configured to present the modified predicted wellness event sequence as a user interface element on the graphical user interface.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the arts to make and use the embodiments.

FIG. 1 is a diagram illustrating an operating environment of an example continuous analyte monitoring system for used with the techniques described herein.

FIG. 2 is a block diagram illustrating an example data receiving device for communicating with a sensor according to some embodiments.

FIG. 3 is a block diagram depicting an example analyte sensor device for communicating with a continuous analyte monitoring system according to some embodiments.

FIG. 4A is a diagram illustrating a system environment of a machine learning architecture for generating predicted wellness event sequences, according to some embodiments.

FIG. 4B is a diagram illustrating an alternative system environment of a machine learning architecture for generating predicted wellness event sequences, according to some embodiments.

FIG. 5 depicts an exemplary predicted wellness event sequence, according to some embodiments.

FIG. 6 is a flowchart illustrating a method for generating and displaying predicted wellness event sequences, according to some embodiments.

FIG. 7 is a flowchart illustrating a method for generating and displaying predicted wellness event sequences including missed predicted wellness event sequences, according to some embodiments.

FIG. 8 is a flow diagram illustrating components of a model training architecture and communications between the components, according to some embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for an improvement patient treatment system through the use of a continuous analyte monitoring system and a prediction model for generating potential patient outcomes based on the continuously monitored analyte levels. The potential patient outcomes may be used to dynamically adjust alerts and notifications and direct patient treatment decisions.

During acute illness or injury, levels of the hormone epinephrine are raised. This leads to the conversion of glucose into lactate, increasing circulating levels. In addition, poor tissue oxygenation, primarily due to a drop in blood pressure, may lead to anaerobic respiration which further contributes to rising lactate levels. Embodiments of the present disclosure utilize continuous lactate (and in some additional embodiments, analyte) monitoring for measuring lactate levels. A prediction model may be updated based on the monitored lactate information which improves the accuracy of the prediction model to predict patient outcomes as well as the capability to dynamically adjust patient treatment. Rises in lactate levels are associated with worse clinical outcomes and could be used to direct treatment decisions. Continuous lactate monitoring could be used to monitor patients, screening for signs of deterioration. This would reduce the blind spot between nursing observations, potentially allowing for earlier identification of patient decline. This could lead to a reduction in patient mortality, reduction in intensive care admission, and shorten inpatient length of stay.

The present disclosure is not limited to glucose as the analyte being continuously monitored. Other analytes, in addition to or alternatively, to glucose may also be utilized as part of the prediction model. These analytes include lactate, ketones, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, or any combination thereof.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

### DEFINITIONS

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

As used herein and in the appended claims, the singular forms “a,” “an,” and “the” include plural referents unless the context clearly dictates otherwise. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20% (e.g., up to 10%, up to 5%, or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the term “measure” and variations thereof can encompass the meaning of a respective term, such as “determine,” “calculate,” and variations thereof.

As used herein, an “analyte” is an enzyme substrate that is subject to be measured or detected. The analyte can be from, for example, a biofluid and can be tested in vivo, ex vivo, or in vitro.

As used herein, a “sensor” is a device configured to detect the presence and/or measure the level of an analyte in a sample, including a continuous analyte sensor which is configured to detect analytes in the sample in a continuous manner.

As used herein, the term “patient” refers to a living animal, and thus encompasses a living mammal and a living human, for example. The term “user” can be used herein as a term that encompasses the term “patient.”

As used herein, the term “machine learning model” refers to a computational framework, which may include pre-trained models adapted through customized inputs (e.g., prompts) or models specifically trained, designed to process data and generate personalized visual elements for display on a user interface.

Generally, embodiments of the present disclosure include systems, devices, and methods for the use of in vivo analyte monitoring systems for multiple different use cases.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system, such as but not limited to wellness, fitness, dietary, research, information or any purposes involving analyte sensing over time. As used herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, or any combination thereof.

Before describing aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. CGM systems, for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (“NFC”) or Radio Frequency Identification (“RFID”) protocol. In vivo analyte monitoring systems can also operate without the need for finger stick calibration.

In vivo analyte monitoring systems can be differentiated from in vitro systems that contact a biological sample outside of the body (or ex vivo) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary Continuous Analyte Monitoring System

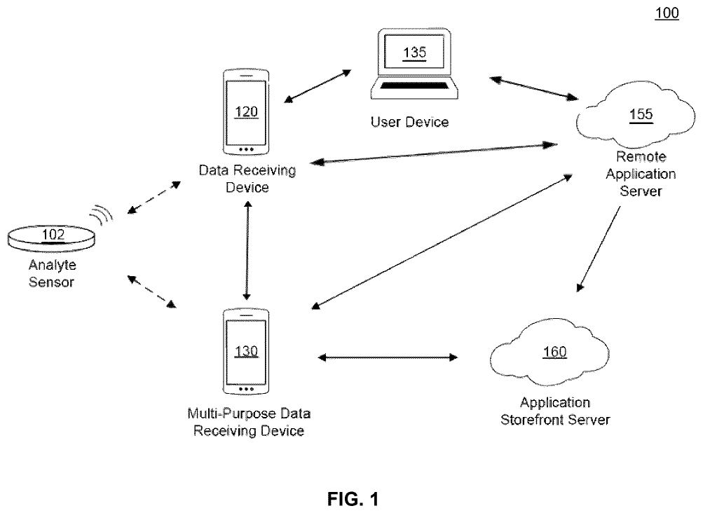

FIG. 1 illustrates an operating environment of a continuous analyte monitoring system 100 capable of embodying the techniques described herein. The continuous analyte monitoring system 100 can include a system of components designed to provide monitoring of parameters, such as analyte levels, of a human or animal body or can provide for other operations based on the configurations of the various components. As embodied herein, the system can include a low-power analyte sensor 102, or simply “sensor” worn by the user or attached to the body for which information is being collected. As embodied herein, the analyte sensor 102 can be a sealed, disposable device with a predetermined active use lifetime (e.g., 1 day, 14 days, 30 days, etc.). Sensors such as analyte sensor 102 can be applied to the skin of the user body and remain adhered over the duration of the sensor lifetime or can be designed to be selectively removed and remain functional when reapplied. The low-power continuous analyte monitoring system 100 can further include a data reading device 120, multi-purpose data receiving device 130, and user device 135 configured as described herein to facilitate retrieval and delivery of data, including analyte data, from the analyte sensor 102.

One or more components of analyte system 100 may be physically located in one or more locations, such as the same location or distributed across different locations. For example, analyte sensor 102 is physically worn by the user who may be located at a home location or in a hospital setting; data receiving device 120 may be located in physical proximity to the analyte sensor 102 (e.g., such as in the same room as the analyte sensor 102) or in a separate physical location but in network communication with the analyte sensor 102. Similarly, other components of analyte system 100 may be located in physical proximity to the analyst sensor 102 or in a separate physical location.

As embodied herein, the continuous analyte monitoring system 100 can include a software or firmware library or application provided, for example via a remote application server 155 or application storefront server 160, to a third-party and incorporated into a multi-purpose data receiving device 130 such as a mobile phone, tablet, personal computing device, or other similar computing device capable of communicating with the analyte sensor 102 over a communication link. Multi-purpose hardware for multi-purpose data receiving device 130 can further include embedded devices, including, but not limited to insulin pumps or insulin pens, having an embedded library configured to communicate with the analyte sensor 102. For example, multi-purpose data receiving device 130 may be a mobile phone or tablet configured to communicate with analyte sensor 102 via an installed application or software program. When the application or software program are uninstalled, multi-purpose data receiving device 130 may be unable to communicate with analyte sensor 102. The installed application may configure multi-purpose data receiving device 130 so that it can process analyte data. Some examples of processing data include displaying the analyte data, analyzing the analyte data, and generating visualizations of the analyte data.

As embodied herein, the continuous analyte monitoring system 100 can further include a data receiving device 120 capable of communicating with analyte sensor 102 over a communication link and multi-purpose data receiving device 130. In contrast to multi-purpose data receiving device 130, data receiving device 120 may be a device dedicated (e.g., without having to install any additional application or software) to communicating with and processing data from analyte sensor 102. In other embodiments, data receiving device 120 may be configured to communicate only with analyte sensor 102 and only process and display data received from and associated with analyte sensor 102.

As embodied herein, the continuous analyte monitoring system 100 can further include user device 135, which may be configured to be in communication with data receiving device 120, multi-purpose data receiving device 130, and/or a remote application server 155. In some embodiments, user device 135 may be implemented as a computer with a fixed location while data receiving device 120 and multi-purpose data receiving device 130 are implemented as mobile computers capable of being transported (e.g., with the patient, with a hospital bed). User device 135 may receive analyte data from data receiving device 120 and multi-purpose data receiving device 130 and provide additional output, input, and processing capabilities for interacting with the analyte data. For example, user device 135 may be implemented as a laptop or a personal computer that includes a keyboard, mouse, and/or a larger screen for displaying the analyte data.

One or more components of continuous analyte monitoring system 100 may be deployed within different physical settings, such as a hospital or home, or different physical use cases, such as patients identified with risk of certain diseases, such as sepsis or congestive heart failure, or have undergone high-risk surgery. Implementation of the components (e.g., data receiving device 120, multi-purpose data receiving device 130, user device 135) may be based on the setting in which continuous analyte monitoring system 100 is deployed. For example, in a hospital setting, one or more data receiving device 120 or multi-purpose data receiving device 130 may be implemented as a patient bedside monitoring device configured to receive and display analyte information provided by analyte sensor 102 and user device 135 may be implemented as a computing device located in a different physical location within the hospital, such as a nurse's station. Remote application server 155 may be configured with modules for storing and configuring electronic medical records (EMR) for patients in one or more hospitals. Remote application server 155 is configured to receive analyte information from user device 135 and/or data receiving device 120 and further configured to update EMR profiles associated with the received analyte information.

Communication between components may also be based on the type of components. For example, the frequency of communicating analyte data between analyte sensor 102 and data receiving device 120 and/or user device 135 may be different from the frequency that the analyte data is communication to the remote application server. For example, data receiving device 120 and/or user device 135, when implemented in a hospital setting, may be utilized to display patient conditions in real-time; accordingly, the frequency of communication between analyte sensor 102 and data receiving device 120 and/or user device 135 may be configured to a high frequency to ensure that analyte data displayed by data receiving device 120 and/or user device 135 is accurate. On the other hand, the frequency of communication between data receiving device 120 and/or user device 135 and remote application server 155 may be set to a lower frequency (i.e., data is transmitted in batches) for storage at the remote application server 155.

Parameters of the communications between components may be dynamically updated based on any number of conditions including the component receiving the analyte data, the type of analyte being monitored, risk factors associated with the patient, time of day, current location of the patient, and a current condition of the patient. For example, there may be configurable threshold settings associated with these conditions that may trigger changes to the communication frequency. As one non-limiting example, a negative change in a patient's condition based on a certain threshold may trigger an increase in communication frequency. As another example, communications during evening hours may be set to a lower frequency then communications during daytime hours.

There may also be location-specific conditions for that dictate the type and frequency of communications. For example, the current location of the patient, such as whether the patient is located at home or within the hospital, may change the types and frequency of messages that are transmitted to the patient's caregiver. As one non-limiting example, when the user is determined to be at home, or otherwise physically located outside of a hospital, the analyte sensor 102 may be configured to communicate directly with remote application server 155 or the caregiver's device (e.g., multi-purpose data receiving device 130 or user device 135), such as when the user is located at home. In this manner, analyte information may be stored directly by the remote application server 155, such as in an EMR associated with the patient, and/or provided directly to the caregiver device even when the patient is located at his home.

Although the illustrated embodiments of the continuous analyte monitoring system 100 include only one of each of the illustrated devices, this disclosure contemplates the continuous analyte monitoring system 100 incorporate multiples of each components interacting throughout the system. For example and without limitation, as embodied herein, data reading device 120 and/or multi-purpose data receiving device 130 can include multiples of each. As embodied herein, multiple data receiving devices 130 can communicate directly with sensor 102 as described herein. Additionally or alternatively, a data receiving device 130 can communicate with secondary data receiving devices 130 to provide analyte data, or visualization or analysis of the data, for secondary display to the user or other authorized parties.

### Exemplary Reader Device

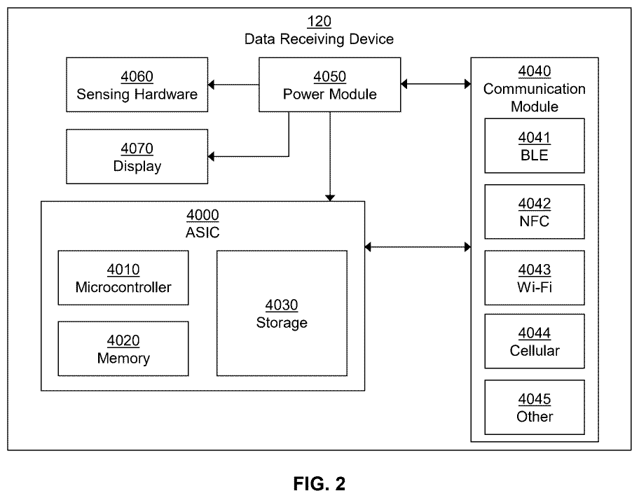

FIG. 2 is a block diagram depicting an example embodiment of a reader device configured as a smartphone. The following description will focus on data receiving device 120 but it is understood that the components and functionality may also apply to other reader devices, such as multi-purpose data receiving device 130 and user device 135. For purpose of illustration and not limitation, reference is made to the exemplary embodiment of data receiving device 120 for use with the disclosed subject matter as shown in FIG. 2. Data receiving device 120 and the related multi-purpose data receiving device 130 include components germane to the discussion of analyte sensor 102 and its operations and additional components can be included. In particular embodiments, data receiving device 120 and multi-purpose data receiving device 130 can be or include components provided by a third party and are not necessarily restricted to include devices made by the same manufacturer as analyte sensor 102.

As illustrated in FIG. 2, data receiving device 120 includes an ASIC 4000 having a microcontroller 4010, memory 4020, and storage 4030 and communicatively coupled with a communication module 4040. Power for the components of data receiving device 120 can be delivered by power module 4050, which as embodied herein can include a rechargeable battery. Data receiving device 120 can further include display 4070 for facilitating review of analyte data received from analyte sensor 102 or other device (e.g., user device 135 or remote application server 155). Data receiving device 120 can include separate user interface components (e.g., physical keys, light sensors, microphones, etc.).

Communication module 4040 can include Bluetooth low energy (BLE) module 4041 and NFC module 4042. Data receiving device 120 can be configured to wirelessly couple with analyte sensor 102 and transmit commands to and receive data from analyte sensor 102. As embodied herein, data receiving device 120 can be configured to operate, with respect to analyte sensor 102 as described herein, as an NFC scanner and a BLE end point via specific modules (e.g., BLE module 4042 or NFC module 4043) of communication module 4040. For example, data receiving device 120 can issue commands (e.g., activation commands for a data broadcast mode of the sensor; pairing commands to identify data receiving device 120) to analyte sensor 102 using a first module of the communication module 4040 and receive data from and transmit data to analyte sensor 102 using a second module of the communication module 4040.

As another example, communication module 4040 can include, for example, cellular radio module 4044. The cellular radio module 4044 can include one or more radio transceivers for communicating using broadband cellular networks, including, but not limited to third generation (“3G”), fourth generation (“4G”), and fifth generation (“5G”) networks. Additionally, communication module 4040 of data receiving device 120 can include Wi-Fi radio module 4043 for communication using a wireless local area network according to one or more of the IEEE 802.11 standards (e.g., 802.11a, 802.11b, 802.11g, 802.11n (aka Wi-Fi 4), 802.11ac (aka Wi-Fi 5), 802.11ax (aka Wi-Fi 6)). Using cellular radio module 4044 or Wi-Fi radio module 4043, data receiving device 120 can communicate with remote application server 155 to receive analyte data or provide updates or input received from a user (e.g., through one or more user interfaces). Communication module 4040 can further include other protocol module 4045 that are compatible with additional wireless standards for Internet of Things (IoT) devices, such as Zigbee and Matter. In some embodiments, other protocol module 4045 can include additional or alternative chipsets for use with other short-range communication schemes, such as short-range radio (other than Bluetooth or BLE), personal area network according to IEEE 802.15 protocols, IEEE 802.11 protocols, infrared communications according to the Infrared Data Association standards (IrDA), etc.

In some embodiments, other protocol module 4045 may also be configured to be compatible with proprietary protocols such as proprietary short-range radio protocols that do not include Bluetooth or BLE. One non-limiting example is the Medical Implement Communication System (MICS) protocol which is configured for transmitting data between medical devices implanted in a patient. MICS protocol operates at a frequency from 402 to 405 MHz. Being compatible with other short-range radio protocols could be particular advantageous in a hospital setting where Bluetooth and/or BLE can potentially interfere with operations and communications with medical devices that are not intended recipients of Bluetooth and/or BLE messages. Other short-range radio protocols avoid this interference issue. Additionally, proprietary short-range radio protocols may also offer more security as Bluetooth and/or BLE can be susceptible to cyberattacks.

As embodied herein, on-board storage 4030 of data receiving device 120 can store analyte data received from analyte sensor 102. Further, data receiving device 120, multi-purpose data receiving device 130, or user device 135 can be configured to communicate with remote application server 155 via a wide area network. As embodied herein, analyte sensor 102 can provide data to data receiving device 120 or multi-purpose data receiving device 130. Data receiving device 120 can transmit the data to user device 135. User device 135 (or multi-purpose data receiving device 130) can in turn transmit that data to remote application server 155 for processing and analysis.

As embodied herein, data receiving device 120 can further include sensing hardware 4060 similar to, or expanded from, sensing hardware 5060 of analyte sensor 102. In particular embodiments, data receiving device 120 can be configured to operate in coordination with analyte sensor 102 and based on analyte data received from the analyte sensor 102. As an example, where analyte sensor 102 is a glucose sensor, data receiving device 120 can be or include an insulin pump or insulin injection pen. In coordination, multi-purpose data receiving device 130 can adjust an insulin dosage for a user based on glucose values received from the analyte sensor.

### Exemplary Sensor Devices

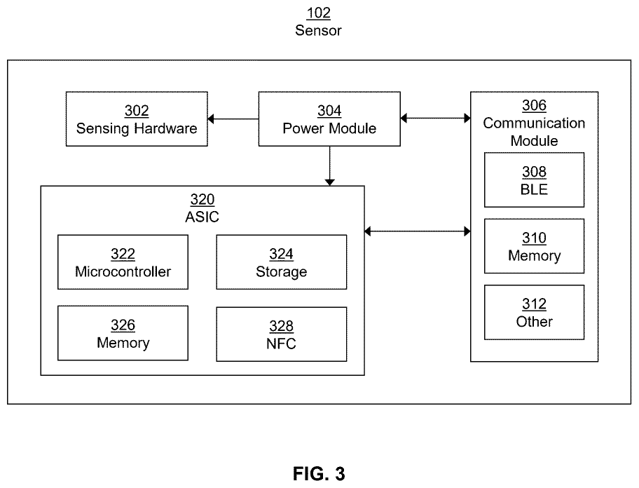

FIG. 3 is a block diagram depicting example embodiments of sensor 102. For purpose of illustration and not limitation, reference is made to the exemplary embodiment of analyte sensor 102 for use with the disclosed subject matter as shown in FIG. 3. FIG. 3 illustrates a block diagram of an example analyte sensor 102 according to exemplary embodiments compatible with the security architecture and communication schemes described herein.

As embodied herein, analyte sensor 102 can include ASIC 320 communicatively coupled with a communication module 306. ASIC 320 can include a microcontroller core 322, on-board memory 326, and storage memory 324. Storage memory 324 can store data used in an authentication and encryption security architecture. Storage memory 324 can store programming instructions for analyte sensor 102. As embodied herein, certain communication chipsets can be embedded in the ASIC 320 (e.g., an NFC transceiver 328). ASIC 320 can receive power from a power module 5050, such as an on-board battery or from an NFC pulse. Storage memory 324 of the ASIC 320 can be programmed to include information such as an identifier for analyte sensor 102 for identification and tracking purposes. Storage memory 324 can also be programmed with configuration or calibration parameters for use by analyte sensor 102 and its various components. Storage memory 324 can include rewritable or one-time programming (“OTP”) memory. The storage memory 324 can be updated using techniques described herein to extend the usefulness of analyte sensor 102.

To perform its functionalities, sensor 102 can further include suitable sensing hardware 302 appropriate to its function. As embodied herein, sensing hardware 302 can include an analyte sensor transcutaneously or subcutaneously positioned in contact with a bodily fluid of a subject. The analyte sensor can generate sensor data containing values corresponding to levels of one or more analytes within the bodily fluid.

The storage memory 324 of the sensor 102 can include the software blocks related to communication protocols of the communication module. For example, the storage memory 324 can include a BLE services software block with functions to provide interfaces to make the BLE module 308 available to the computing hardware of the sensor 102. These software functions can include a BLE logical interface and interface parser. BLE services offered by the communication module 306 can include the generic access profile service, the generic attribute service, generic access service, device information service, data transmission services, and security services. The data transmission service can be a primary service used for transmitting data such as sensor control data, sensor status data, analyte measurement data (historical and current), and event log data. The sensor status data can include error data, current time active, and software state. The analyte measurement data can include information such as current and historical raw measurement values, current and historical values after processing using an appropriate algorithm or model, projections and trends of measurement levels, comparisons of other values to patient-specific averages, calls to action as determined by the algorithms or models and other similar types of data.

As embodied herein, communication module 306 of sensor 102 can be implemented as or include one or more modules to support analyte sensor 102 communicating with other devices of the continuous analyte monitoring system 100. As an example only and not by way of limitation, example communication module 306 can include BLE module 308, memory 310, and other protocol module 312. As used throughout this disclosure, BLE refers to a short-range communication protocol optimized to make pairing of Bluetooth devices simple for end users. Communication module 306 can transmit and receive data and commands via interaction with similarly-capable communication modules of data receiving device 120 or user device 135. Communication module 306 may store the data and commands in memory 310. Communication module 306 can include an additional module 312 for other protocols and/or additional or alternative chipsets for use with similar short-range communication schemes, such as a personal area network according to IEEE 802.15 protocols, IEEE 802.11 protocols, infrared communications according to the Infrared Data Association standards (IrDA), etc.

According to aspects of the disclosed subject matter, and as embodied herein, a sensor 102 can be configured to communicate with multiple devices concurrently by adapting the features of a communication protocol or medium supported by the hardware and radios of the sensor 102. As an example, the BLE module 308 of the communication module 306 can be provided with software or firmware to enable multiple concurrent connections between the sensor 102 as a central device and the other devices such as anyone of data receiving device 120, multi-purpose data receiving device 130, and/or user device 135.

Connections, and ensuing communication sessions, between two devices using a communication protocol such as BLE can be characterized by a similar physical channel operated between the two devices (e.g., a sensor 102 and data receiving device 120). The physical channel can include a single channel or a series of channels, including for example and without limitation using an agreed upon series of channels determined by a common clock and channel- or frequency-hopping sequence. Communication sessions can use a similar amount of the available communication spectrum, and multiple such communication sessions can exist in proximity. In certain embodiment, each collection of devices in a communication session uses a different physical channel or series of channels, to manage interference of devices in the same proximity.

For purpose of illustration and not limitation, reference is made to an exemplary embodiment of a procedure for a sensor-receiver connection for use with the disclosed subject matter. First, the sensor 102 repeatedly advertises its connection information to its environment in a search for a data receiving device 120. The sensor 102 can repeat advertising on a regular basis until a connection established. The data receiving device 120 detects the advertising packet and scans and filters for the sensor 102 to connect to through the data provided in the advertising packet. Next, data receiving device 120 sends a scan request command and the sensor 102 responds with a scan response packet providing additional details. Then, the data receiving device 120 sends a connection request using the Bluetooth device address associated with the data receiving device 120. The data receiving device 120 can also continuously request to establish a connection to a sensor 102 with a specific Bluetooth device address. Then, the devices establish an initial connection allowing them to begin to exchange data. The devices begin a process to initialize data exchange services and perform a mutual authentication procedure.

During a first connection between the sensor 102 and data receiving device 120, the data receiving device 120 can initialize a service, characteristic, and attribute discovery procedure. The data receiving device 120 can evaluate these features of the sensor 102 and store them for use during subsequent connections. Next, the devices enable a notification for a customized security service used for mutual authentication of the sensor 102 and data receiving device 120. The mutual authentication procedure can be automated and require no user interaction. Following the successful completion of the mutual authentication procedure, the sensor 102 sends a connection parameter update to request the data receiving device 120 to use connection parameter settings preferred by the sensor 102 and configured to maximum longevity.

In some embodiments, the data receiving device 120 then performs sensor control procedures to backfill historical data, current data, event log, and factory data. As an example, for each type of data, the data receiving device 120 sends a request to initiate a backfill process. The request can specify a range of records defined based on, for example, the measurement value, timestamp, or similar, as appropriate. The sensor 102 responds with requested data until all previously unsent data in the memory of the sensor 102 is delivered to the data receiving device 120. The sensor 102 can respond to a backfill request from the data receiving device 120 that all data has already been sent. Once backfill is completed, the data receiving device 120 can notify sensor 102 that it is ready to receive regular measurement readings. The sensor 102 can send readings across multiple notifications result on a repeating basis. As embodied herein, the multiple notifications can be redundant notifications to ensure that data is transmitted correctly. Alternatively, multiple notifications can make up a single payload.

As embodied herein, certain calibration features for the sensing hardware 302 of the analyte sensor 102 can be adjusted based on external or interval environment features as well as to compensate for the decay of the sensing hardware 302 during expended period of disuse (e.g., a “shelf time” prior to use). The calibration features of the sensing hardware 302 can be autonomously adjusted by the sensor 102 (e.g., by operation of the ASIC 320 to modify features in the memory 326 or storage memory 324) or can be adjusted by other devices of the CAM system 100.

As an example, sensor sensitivity of the sensing hardware 302 can be adjusted based on external temperature data or the time since manufacture. When external temperatures are monitored during the storage of the sensors, the disclosed subject matter can adaptively change the compensation to sensor sensitivity over time when the device experiences changing storage conditions. For purpose of illustration not limitations, adaptive sensitivity adjustment can be performed in an “active” storage mode where the analyte sensor 102 wakes up periodically to measure temperature. These features can save the battery of the analyte device and extend the lifespan of the analyte sensors. At each temperature measurement, the analyte sensor 102 can calculate a sensitivity adjustment for that time period based on the measured temperature. Then, the temperature-weighted adjustments can be accumulated over the active storage mode period to calculate a total sensor sensitivity adjustment value at the end of the active storage mode (e.g., at insertion). Similarly, at insertion, the analyte sensor 102 can determine the time difference between manufacture of the sensor 102 (which can be written to the storage memory 324 of the ASIC 320) or the sensing hardware 302 and modify sensor sensitivity or other calibration features according to one or more known decay rates or formulas.

Additionally, for purpose of illustration and not limitation, as embodied herein, sensor sensitivity adjustments can account for other sensor conditions, such as sensor drift. Sensor sensitivity adjustments can be hardcoded into the analyte sensor 102 during manufacture, for example in the case of sensor drift, based on an estimate of how much an average sensor would drift. Analyte sensor 102 can use a calibration function that has time-varying functions for sensor offset and gain, which can account for drift over a wear period of the sensor. Thus, analyte sensor 102 can utilize a function used to transform an interstitial current to interstitial glucose utilizing device-dependent functions describing analyte sensor 102 drift over time, and which can represent sensor sensitivity, and can be device specific, combined with a baseline of the glucose profile. Such functions to account for sensor sensitivity and drift can improve analyte sensor 102 accuracy over a wear period and without involving user calibration.

In some embodiments, the sensor 102 detects raw measurement values from sensing hardware 302. On-sensor processing can be performed, such as by one or more models trained to interpret the raw measurement values. Models can be machine learned models trained off-device to detect, predict, or interpret the raw measurement values to detect, predict, or interpret the levels of one or more analytes. Additional trained models can operate on the output of the machine learning models trained to interact with raw measurement values. As an example, models can be used to detect, predict, or recommend events based on the raw measurements and type of analyte(s) detected by the sensing hardware 302. Events can include, initiation or completion of physical activity, meals, application of medical treatment or medication, emergent health events, and other events of a similar nature.

Models can be provided to any combination of the sensor 102, data receiving device 120, or multi-purpose data receiving device 130 during manufacture or during firmware or software updates. Models can be periodically refined, such as by the manufacturer of the sensor 102 or the operator of the CAM system 100, based on data received from the sensor 102 and data receiving devices of an individual user or multiple users collectively. In certain embodiments, the sensor 102 includes sufficient computational components to assist with further training or refinement of the machine learned models, such as based on unique features of the user to which the sensor 102 is attached. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The models can also include algorithmic or rules-based models in addition to machine learned models. Model-based processing can be performed by other devices, including the data receiving device 120 or multi-purpose data receiving device 130, upon receiving data from the sensor 102 (or other downstream devices).

When applied to continuous lactate monitoring, machine learning models may rely on the real-time data being provided by, for example, sensor 102, to continuously train the model or update the training set for the model. Machine learning models of the present disclosure may also be trained using patient vital information, patient medical history, and in some embodiments, cohort information, which would increase the size of the training set to include lactate and medical information from patients that are similar to the current patient.

In some embodiments, machine learning models applied to a continuous analyte monitoring system are configured to classify each analyte measurement (or more generally, a portion of a spike, such as a spike start, a spike end, and/or a spike peak) as being part of a spike (or not). A spike is an example of a predicted wellness event and the spike characteristics, such as spike start, spike end, spike peak, are event indications. In some embodiments, machine learning models may be trained to perform this classification function of events and event indications with incomplete data from a sensor of the continuous analyte monitoring system, e.g., before the spike ends. Accordingly, the combination of a machine learning model and a continuous analyte monitoring system is configured to support near real-time applications, where the model may notify users of medical information, e.g., related to an ongoing spike.

Machine learning models of the present disclosure therefore provide an improvement to conventional machine learning models used for event detection, which typically require complete data in order to perform any detection (i.e., spikes have ended).

Data transmitted between the sensor 102 and any one of a data receiving device 120, multi-purpose data receiving device 130, and/or user device 135 can include raw or processed measurement values. Data transmitted between the sensor 102 and any one of a data receiving device 120, multi-purpose data receiving device 130, and/or user device 135 can further include alarms or notification for display to a user. The data receiving device 120, multi-purpose data receiving device 130, and/or user device 135 can display or otherwise convey notifications to the user based on the raw or processed measurement values or can display alarms when received from the sensor 110. Alarms that may be triggered for display to the user include alarms based on the predicted patient outcome (e.g., when compared to different levels of severity thresholds), direct analyte values (e.g., one-time reading exceeding a threshold or failing to satisfy a threshold), analyte value trends (e.g., average reading over a set period of time exceeding a threshold or failing to satisfy a threshold; slope); analyte value predictions (e.g., algorithmic calculation based on analyte values exceeds a threshold or fails to satisfy a threshold), sensor alerts (e.g., suspected malfunction detected), communication alerts (e.g., no communication between sensor 102 and data receiving device 120 for a threshold period of time; unknown device attempting or failing to initiate a communication session with the sensor 102), reminders (e.g., reminder to charge data receiving device 120; reminder to take a medication or perform other activity), and other alerts of a similar nature. For purpose of illustration and not limitation, as embodied herein, the alarm parameters described herein can be configurable by a user or can be fixed during manufacture, or combinations of user-settable and non-user-settable parameters.

### Exemplary Over-The-Air Updates

FIG. 1 further illustrates an example operating environment for providing over-the-air (“OTA”) updates for use with the techniques described herein. An operator of continuous analyte monitoring system 100 can bundle updates for data receiving device 120 or analyte sensor 102 into updates for an application executing on multi-purpose data receiving device 130. Using available communication channels between data receiving device 120, multi-purpose data receiving device 130, and analyte sensor 102, multi-purpose data receiving device 130 can receive regular updates for data receiving device 120 or analyte sensor 102 and initiate installation of the updates on data receiving device 120 or analyte sensor 102. Multi-purpose data receiving device 130 acts as an installation or update platform for data receiving device 120 or analyte sensor 102 because the application that enables the multi-purpose data receiving device 130 to communicate with analyte sensor 102, data receiving device 120 and/or remote application server 155 can update software or firmware on data receiving device 120 or analyte sensor 102 without wide-area networking capabilities.

As embodied herein, remote application server 155 operated by the manufacturer of analyte sensor 102 and/or the operator of continuous analyte monitoring system 100 can provide software and firmware updates to the devices of continuous analyte monitoring system 100. In particular embodiments, remote application server 155 can provides the updated software and firmware to user device 135 or directly to a multi-purpose data receiving device. As embodied herein, remote application server 155 can also provide application software updates to application storefront server 160 using interfaces provided by the application storefront. Multi-purpose data receiving device 130 can contact the application storefront server 160 periodically to download and install the updates.

After multi-purpose data receiving device 130 downloads an application update including a firmware or software update for data receiving device 120 or analyte sensor 102, data receiving device 120 or analyte sensor 102 and multi-purpose data receiving device 130 establish a connection. Multi-purpose data receiving device 130 determines that a firmware or software update is available for data receiving device 120 or analyte sensor 102. Multi-purpose data receiving device 130 can prepare the software or firmware update for delivery to data receiving device 120 or analyte sensor 102. As an example, multi-purpose data receiving device 130 can compress or segment the data associated with the software or firmware update, can encrypt or decrypt the firmware or software update, or can perform an integrity check of the firmware or software update. Multi-purpose data receiving device 130 sends the data for the firmware or software update to data receiving device 120 or analyte sensor 102. Multi-purpose data receiving device 130 can also send a command to data receiving device 120 or analyte sensor 102 to initiate the update. Additionally or alternatively, multi-purpose data receiving device 130 can provide a notification to the user of multi-purpose data receiving device 130 and include instructions for facilitating the update, such as instructions to keep data receiving device 120 and multi-purpose data receiving device 130 connected to a power source and in close proximity until the update is complete.

Data receiving device 120 or analyte sensor 102 receives the data for the update and the command to initiate the update from multi-purpose data receiving device 130. Data receiving device 120 can then install the firmware or software update. To install the update, data receiving device 120 or analyte sensor 102 can place or restart itself in a so-called “safe” mode with limited operational capabilities. Once the update is completed, data receiving device 120 or analyte sensor 102 re-enters or resets into a standard operational mode. Data receiving device 120 or analyte sensor 102 can perform one or more self-tests to determine that the firmware or software update was installed successfully. Multi-purpose data receiving device 130 can receive the notification of the successful update. Multi-purpose data receiving device 130 can then report a confirmation of the successful update to remote application server 155.

In particular embodiments, storage memory 324 of analyte sensor 102 includes OTP memory. The term OTP memory can refer to memory that includes access restrictions and security to facilitate writing to particular addresses or segments in the memory a predetermined number of times. Storage memory 324 can be prearranged into multiple pre-allocated memory blocks or containers. The containers are pre-allocated into a fixed size. If storage memory 324 is OTP memory, the containers can be considered to be in a non-programmable state. Additional containers which have not yet been written to can be placed into a programmable or writable state. Containerizing storage memory 324 in this fashion can improve the transportability of code and data to be written to storage memory 324. Updating the software of a device (e.g., the sensor device described herein) stored in an OTP memory can be performed by superseding only the code in a particular previously-written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

### Summary of Machine Learning Model for Wellness Events

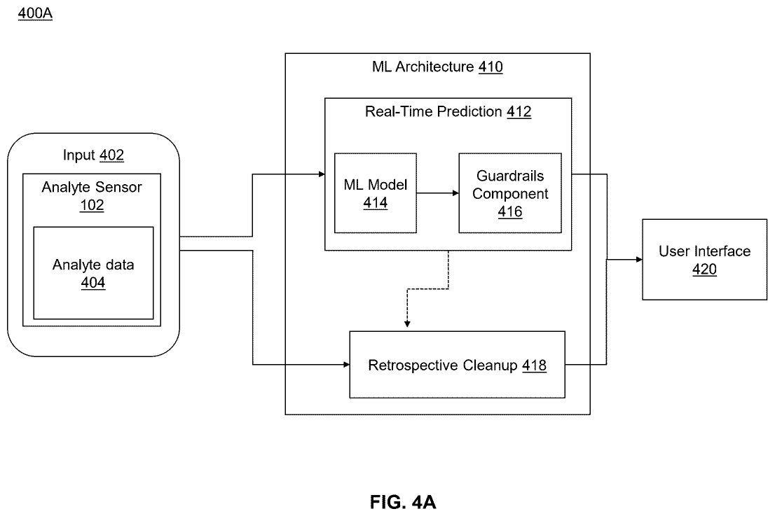

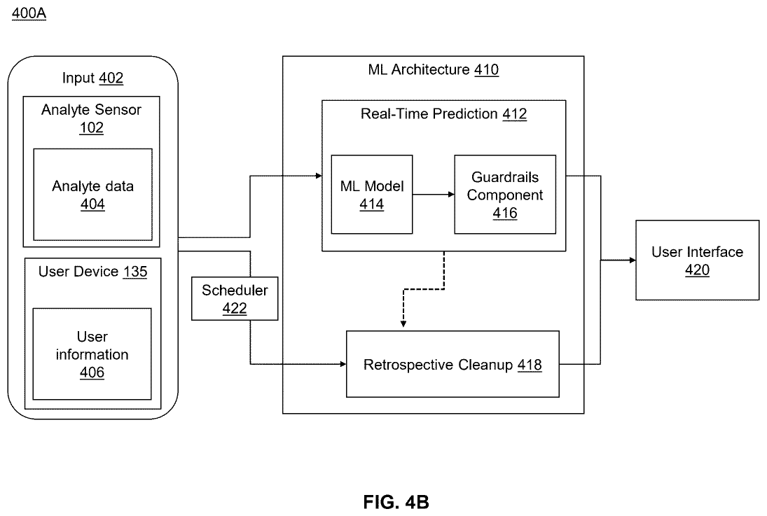

FIG. 4A is a block diagram of an exemplary machine learning system 400A for generating predicted wellness events based user analyte data. In some embodiments, one or more components of machine learning system 400 may be implemented in continuous analyte monitoring (CAM) system 100, according to some embodiments.

For example, machine learning system 400 may include a machine learning architecture 410 that includes a real-time prediction component 412 and a retrospective cleanup component 418. In some embodiments, machine learning architecture 410 may be implemented in one or more components of CAM system 100, such as data receiving device 120, user device 135, multi-purpose data receiving device 130, and remote application server 155. In some embodiments, various components of machine learning architecture 410 may be distributed across one or more of components of CAM system 100. As a non-limiting example, real-time prediction component 412 may be implemented in one component of CAM system 100 and retrospective clean component 418 may be implemented in a different component of CAM system 100. In some embodiments, machine learning architecture 410 may be fully implemented in one component of CAM system 100.

In one particular example, real-time prediction component 412 and retrospective cleanup component 418 is implemented in a server such as remote application server 155 and user interface 420 is implemented as part of an application installed on a user device such as, data receiving device 120, user device 135, or multi-purpose data receiving device 130. In another example, components of machine learning architecture 410 may be distributed between the server and the user device. For example, real-time prediction component 412, installed on the user device, may communicate with remote components of machine learning architecture 410, such as retrospective clean component 418. As another example, real-time prediction component 412 may be distributed between the user device and the server, such as the machine learning model 414 implemented on the user device while guardrail component 416 is implemented on the server, or a lightweight version of machine learning model 414 may be implemented on the user device while a heavyweight version of machine learning model may be implemented on the server.

Machine learning system 400 may continuously provide input 402 to machine learning architecture 410. In some examples, input 402 may be received from analyte sensor 102 at a predetermined cadence, such as every five minutes. The cadence at which input 402 is provided to machine learning architecture 410 is different from the cadence at which analyte sensor 102 generates analyte data (e.g., every five minutes versus every five seconds). In this example, analyte data from analyte sensor 102 may be aggregated until input 402 is provided to machine learning architecture 410. In other examples, the cadence at which input 402 is provided to machine learning architecture 410 may be the same such that analyte data from analyte sensor 102 can be provided in real-time to machine learning model 414.

Input 402 may be provided from one or more sources within machine learning system 400. For example, analyte sensor 102 may provide real-time analyte data 404 of patients (e.g., received from analyte sensor 102) as input to machine learning architecture 410, which is configured to process the real-time analyte data and generate predictions related to wellness events for based on the real-time analyte data. In some embodiments, machine learning architecture generates a predicted wellness event sequence comprising one or more wellness events depicted over a predefined time period. Predictions of wellness event include both the event itself (e.g., a spike) and the event indications of the event (e.g., spike start, spike end, spike peak, intra-spike peak, intra-spike dip).

Analyte sensor 102 may be implemented as a biowearable analyte (e.g., lactate, glucose, dual) sensor. Benefits of using a biowearable sensor include but are not limited to being less expensive, non-invasive/non-permanent, no procedure/no in-person visit, no expert team and equipment, easy start-up, easy data capture, easy data sharing with patient and provider. Additionally, a biowearable sensor may be updated with new hardware and/or software to provide longer-term improvements including providing an accelerometer/altimeter for activities of daily living, providing heart rate and heart rate variability data capture, extending wear-time from 15 days to one month, and adding additional biomarkers including proteins.

Analyte data 404 associated with a patient (e.g., from analyte sensor 102) may be provided to machine learning architecture 410. Analyte data 404 may include any analyte including, but not limited to, lactate, glucose, or creatinine. Any combination of analytes may be utilized and may depend on the use-case deployment for which the predicted patient outcome is being provided.

In some embodiments, real-time prediction component 412 may include a machine learning model 414 and a guardrail component 416. Machine learning model 414 is configured to receive analyte data 404 and generate predicted wellness events based on the analyte data. Examples of analyte data 404 include but are not limited glucose, ketone, alcohol, and lactate. Examples of wellness events that can be generated from real-time analyte data include but are not limited to glucose spikes, glucose crashes, sensor drop outs, temperature-based events, ketone-based events, alcohol-based events, and lactate-based events (e.g., health deterioration related to a disease such as heart failure or sepsis).

In some embodiments, machine learning model 414 generates a predetermined number of predicted wellness events for a predetermined time period that is represented by analyte data 404. Each predicted wellness event may be associated with a point in time, where each point in time may be within the predetermined period of time. As a non-limiting example, analyte data 404 may be data aggregated by analyte sensor 102 for a predetermined time period, such as five minutes, and machine learning model 414 may generate a one or more predicted wellness events within the time period. As one example, the number of predicted wellness events may be implemented as a predetermined number of predicted wellness events, such as six, within that time period. In some embodiments, real-time prediction component 412 may provide one of the predicted wellness events out of the predetermined number for display by graphical user interface 420. In embodiments where the predicted wellness event is a predicted glucose event, such as a glucose crash or a glucose spike, real-time prediction component 412 may provide one of the predicted glucose events to graphical user interface 420 for display as a user interface element in a visual interface.

In some embodiments, machine learning model 414 generates predicted wellness events (e.g., glucose spikes, glucose crashes) by analyzing a number of data points in relation to a specific point in time in the analyte data 404. The number of data points may be predetermined. As one non-limiting example, machine learning model 414 evaluates eighteen past data points preceding the current time and five future data points succeeding the current time to determine whether a wellness event has occurred at the current time in which machine learning model 414 is generating a prediction. The number of data points may also be time dependent. As another non-limiting example, machine learning model 414 evaluates past data points that are available in a first time window 15 minutes prior to the current time and future data points that are available in a second time window 5 minutes after the current time. In some embodiments, machine learning model 414 is operating on a time delayed basis such that there may be a delay period between the time in displaying the predicted wellness event on graphical user interface 420 and the time at which the predicted wellness event is predicted to have occurred. This delay period may be a predetermined time period that is determined by the amount of future data points that are included in the prediction or by a predetermined time. For example, graphical user interface 420 may display analyte data and/or predicted wellness events that occurred twenty five minutes prior to the present time. In this manner, the current time at which machine learning model 414 is generating predicted wellness events is not necessarily the present time (i.e., the time at which a user is viewing graphical user interface 420). Accordingly, future data points is in relation to the current time that the machine learning model 414 is generating predicted wellness events.

The timing for generating predicted wellness events may be different from the timing at which predicted wellness events are presented on graphical user interface 420. Machine learning architecture 410 may be configured to generate predictions based on a window of data both before and after a time of a predicted wellness event. The amount of time before and after may be a predetermined time period or a predetermined number of data points preceding the time of the predicted wellness event. Upon request or a scheduled event, machine learning architecture 410 may process analyte data 404 for a time period. Real-time prediction component 412 and guardrail component 416 may then identify potential wellness events for times within the time period using the window of data before and after a time of a potential wellness event.

Machine learning architecture 410 may be configured to update the graphical user interface 420 with annotations to indicated the predicted wellness event. As one example, graphical user interface 420 may initially display analyte levels of a user for a certain period without any annotations indicating a predicted wellness event. After generating predicted wellness events, machine learning architecture 410 may then update graphical user interface 420 by, for example, annotating the analyte levels to indicate any predicted wellness events. The annotations may be configured to indicate the time of the predicted wellness event and any user contextual data for the predicted wellness event (e.g., whether the time coincides with user activity such as a meal or exercise).

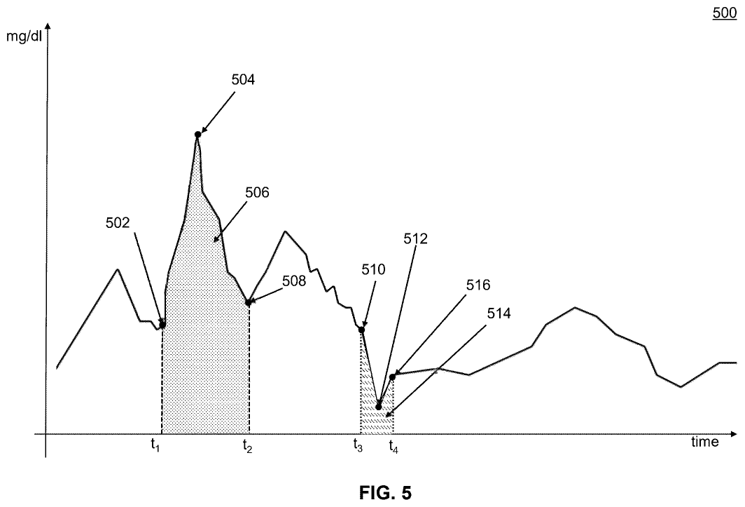

In some embodiments, machine learning model 414 may provide the predicted wellness events to guardrail component 416 for additional processing. Guardrail component 416 is configured to analyze or review a predicted wellness event sequence to identify predicted wellness events in the sequence that may be an inaccurate prediction. ML model 414 may periodically generate inaccurate predictions due to data quality issues like missing datapoints in input sequence, model drift, or model hallucination. An exemplary predicted wellness event sequence is discussed with respect to FIG. 5 below. In some embodiments, output from machine learning model 414 may be considered a predicted wellness event sequence that comprises one or more predicted wellness events identified by machine learning model. Guardrail component 416 is configured to identify any false positive events or a false negative events in the predicted wellness event sequence. Guardrail component 416 may further be configured to modify the predicted wellness event sequence based on the presence of at least one of the false positive event or the false negative event. An example of a false positive event is a false glucose spike and an example of a false negative event is a false glucose crash. An example of a predicted wellness event sequence are a time-ordered series of predicted glucose crashes or spikes. An exemplary predicted wellness event sequence is described with respect to FIG. 5. Examples of guardrail component 416 modifying the predicted wellness event sequence including removing a false glucose spike and removing a false negative event from the predicted wellness event sequence.

In some embodiments, guardrail component 416 is implemented where machine learning model 414 is configured to classify one portion of a spike at a time. Because machine learning model 414 may be used in some embodiments to only predict whether a current glucose reading is in or out of a spike, guardrail component 416 may be needed to ensure that the predictions from machine learning model 414 are consistent over the span of a predicted spike. Because machine learning model 414 is generating predictions based on incomplete analyte data, the role of guardrail component 416 is to confirm the predictions. That is, guardrail component 416 ensuse that individual glucose measurements associated with a spike are classified consistently. The use of guardrail component 416 in machine learning system 400 therefore differs from prior art system that detect/classify entire spikes.

In some embodiments, guardrail component 416 may be implemented as a rules-based wrapper for the output from machine learning model 414. Guardrail component may be configured with rules and threshold conditions that are applied to output of machine learning model 414 to confirm or otherwise test predictions from machine learning model 414. As one example, a rule installed within guardrail component 416 may specify that if a predicted spike comprises less than 6 data points to predict the spike, then guardrail component 416 may be configured to mute the predicted spike. And if the number of data points is greater than 6, then guardrail component may be configured to look at the area under the curve for past the data points and compute a ratio. If the computed ratio is greater than a predetermined threshold amount, then guardrail component 416 may be configured to continue the spike and if the computed ratio is less than the predetermined threshold amount, then guardrail component 416 may be configured to mute the spike.

Guardrail component 416 may be configured to process analyte data 404 to analyze the predicted spikes from machine learning model 414 to confirm or change the classification based on certain conditions. For example, guardrail component 416 may be configured to change a prediction of “out of spike” to “in spike” when it sees that the previous 6 predictions were all “in spike.”

In some embodiments, real-time prediction component 412 may be implemented without guardrail component 416 (e.g., the functionality of guardrail component 416 may be integrated directly into machine learning model 414, or machine learning model 414 may be trained with sufficient data to mitigate the need to perform additional processing of its output). In such embodiments, output of real-time prediction component 412 may be provided directly by machine learning model 414.

Although only one machine learning architecture 410 is depicted in FIG. 4A, CAM system 100 may utilize more than one machine learning architecture 410 for different characteristics. For example, there may be a machine learning architecture 410 generated and trained to generate predicted wellness event sequences based on user cohort (e.g., any one or combination of age, sex, disease, patient history, just to name a few examples). As another example, CAM system 100 may implement machine learning architecture 410 dedicated to generating predictions for female users, one for generating predictions for male users, one for generating predictions for female users under 40, and one for female users over 40.

In some embodiments, machine learning model 414 may be trained using supervised learning. In such embodiments, each training sample used for training may be a series of glucose values (e.g., a current value being classified, 18 previous values, and 5 subsequent values). Each training sample also may have a ground truth label indicating whether the current value is in a spike or not. During each training iteration, the machine learning model 414 may process a training sample and generate a prediction on whether the current value is in a spike. The training algorithm may compare the prediction to the ground truth label of the training sample according to a loss function and, based on the result of the comparison, update machine learning model 414. Training may continue iteratively until a terminating condition is met. For example, training could continue until the differences between the model's predictions and the ground truth labels are within a threshold range. As another example, training could terminate after a sufficiently large number of training iterations have been completed.

In some embodiments, machine learning architecture 410 may implemented as software installed on user device, such as a mobile phone, tablet, or wearable device. In such embodiments, machine learning architecture 410 may be customized based on user information and downloaded and installed as software on a user device (e.g., responsive to user request).

In another example embodiment, machine learning architecture 410 may include a retrospective update component 418 to provide additional information to be displayed with or integrated into the predicted wellness event sequence output by real-time prediction component 412. Retrospective update component 418 may increase the accuracy of the predicted wellness event sequence by identifying any wellness events that are missing (i.e., failed to be predicted) from the by the predicted wellness event sequence output by real-time prediction component 412.

In some embodiments, retrospective update component 418 receives analyte data 404 on a schedule different from how often analyte data 404 is provided to real-time prediction component 412. That is, real-time prediction component 412 may receive analyte data 404 aggregated for a different predetermined time period than retrospective update component 418 receives analyte data 404. For example, real-time prediction component 412 may receive analyte data 404 collected and delivered in five minute increments. Machine learning model 414 may therefore generate predictions based on the analyte data 404 from a predetermined time period, such as five minutes. Accuracy of the predicted wellness event sequences may be increased by decreasing the predetermined time period, any amount down to 1 minute, of analyte data 404 that is provided as input to machine learning model 414.

In some embodiments, retrospective update component 418 further supports machine learning model 414 in its predictions based on incomplete data. As noted above, the machine learning model 414 generates predictions (e.g., determining whether a current glucose reading is in/out of spike) without the benefit of seeing the entire data (i.e., the current spike hasn't concluded yet). As a non-limiting example, analyte data 404 may include 18 past data points and 5 future data points that could indicate that a portion of a spike that is just about to start. The term “future” means data points that are subsequent in time to the point in time where machine learning model 414 is generating a prediction based on the analyte data 404. Machine learning model 414 is tasked with generating predictions based on this incomplete data and may not have the benefit of complete data points for the spike. Accordingly, predictions of machine learning model 414 may have inaccuracies (i.e., false positives, false negatives). To address the inaccuracies of the predictions from machine learning model 414, retroactive update component 418 retroactively reviews the complete data points to detect spikes and evaluate the predictions from machine learning model 414. In some embodiments, retroactive update component 418 could be implemented based on rules (e.g., a spike is detected by finding local minima, maxima, slopes, etc. in analyte data 404).

The difference in the predetermined time periods is based on the different roles of real-time prediction component 412 and retrospective update component 418. Real-time prediction component 412 may include machine learning model 414 that is trained to generate output, such as a predicted wellness event sequence, for real-time or near-real time display on graphical user interface 420. Real-time prediction component 412 may therefore be configured to receive analyte data 404 aggregated over a shorter time period in order to generate the output more quickly. Retrospective update component 418 may be configured to identify any missed wellness events that were missed by real-time prediction component 412. The lack of accuracy of real-time prediction component 412 may be caused by the reduced data due to the shortened time frame that analyte data 404 is aggregated. In this manner, real-time prediction component 412 is configured to process an increased amount of data, such as analyte data 404 aggregated over a longer time window, such as one hour, six hours, twelve hours, or twenty four hours.

In some embodiments, graphical user interface 420 is configured to display, in time-order, all received predicted wellness events sequences for a predetermined time window. Predicted wellness events may be displayed as user interface elements, such as particular icons, on graphical user interface 420, in relation where that predicted wellness event occurs in the predicted wellness event sequence.

In some embodiments, machine learning architecture 410 modifies its state by feeding previous input into the machine learning model 414 to improve on future predictions. This modification may be based on its predicted wellness events by providing the predicted wellness events, as well as any corrections by guardrail component 416 and/or retrospective update component 418 as input to machine learning model 414 in order to improve the accuracy of machine learning model 414, and reduce the need for either guardrail component 416 and/or retrospective update component 418 in subsequent iterations of machine learning model 414. That is, machine learning architecture 410 may be continuously utilize corrections from guardrail component 416 and/or retrospective update component 418 to determine the accuracy of prior outcome predictions and provide continued training of machine learning model 414 so as to improve the accuracy of future predictions.

ML involves computers discovering how they can perform tasks without being explicitly programmed to do so. ML includes, but is not limited to, artificial intelligence, deep learning, fuzzy learning, supervised learning, unsupervised learning, etc. Machine learning algorithms may build an initial prediction model based on sample data, known as “training data”, in order to make predictions or decisions without being explicitly programmed to do so. In some embodiments, this sample data includes analyte data from any number of patients and may be provided by any number of CAM systems. For supervised learning, the computer is presented with example inputs and their desired outputs and the goal is to learn a general rule that maps inputs to outputs. In another example, for unsupervised learning, no labels are given to the learning algorithm, leaving it on its own to find structure in its input. Advantages of unsupervised learning include automatically discovering trends or patterns in data, or a means towards another goal, such as improved accuracy of future predictions.

A machine-learning engine may use various classifiers to map concepts or data provided by different CAM systems and/or different deployment to capture relationships between concepts (e.g., analyte levels and patient outcomes in different deployments) and an accuracy of prior predicted patient outcomes. The classifier (discriminator) is trained to distinguish (recognize) in variations of data from different patients, CAM systems, and/or different deployments.

In some aspects, machine learning models are trained on a remote machine learning platform using a history of analyte data and patient information from one or more other patients, CAM systems, and/or different deployments. In addition, collecting patient data into large training sets may allow for the training data to be normalized (e.g., not skewed by a single or few occurrences of a data artifact). In one embodiment, prediction models are continuously updated as new patient information is received.

An embodiment of training machine learning model 414 is now discussed. In some embodiments, machine learning model 414 may be implemented as a recurrent neural network (RNN), such as a Long Short-Term Memory (LSTM) network, for learning sequential data by capturing both short-term and long-term dependencies within glucose datasets. This allows machine learning model 414 to recognize not only individual glucose values but also contextual relationships of glucose values to surrounding glucose values, such as how a small dip following a rise might indicate an intra-spike dip rather than the true end of a glucose spike.

Machine learning model 414 can be initially trained via supervised learning using collected training data that contains labeled spikes and patterns. Examples of such labels include spike start, spike end, spike peak, intra-spike dip, and intra-spike peak. Spike start, spike end, spike peak, intra-spike dip, and intra-spike peak are examples of event indications or characteristics of a particular wellness event, a glucose spike. A spike start event indication represents a recognized start to a glucose spike within the training data; a spike end event indication represents a recognized end to the glucose spike; a spike peak event indication represents the recognized peak of the glucose level within the training data; an intra-spike dip event indication represents a temporary low point (i.e., followed by a temporary rise) in glucose level during a spike event, which does not mark the recognized end to the glucose spike (i.e., a dip internal to the spike event); and an intra-spike rise event indication represents a temporary high point (i.e., followed by a temporary fall) in glucose levels during a spike event, which does not mark the recognized peak in glucose levels.

Relevant features are selected or engineered from the dataset to improve model performance. Feature selection techniques are used to identify which attributes (features) have the most influence on the target variable. In embodiments, various feature selection techniques can be employed, including but not limited to filter methods such as information gain, chi-square test, Fisher's score, correlation coefficient, variance threshold, and mean absolute difference. Wrapper methods like forward feature selection, backward feature elimination, and exhaustive feature selection can also be used. Embedded methods, including lasso regularization and random forest importance, as well as recursive feature elimination, are other viable options. These techniques help in identifying the most relevant features, thereby enhancing the accuracy and efficiency of the model. However, not all methods may be suitable for the specific data of the invention, and the choice of technique should be based on the nature and characteristics of the dataset.

Feature engineering can also create new features through transformations or combinations of existing ones. In some embodiments, synthetic spikes may be generated based on the labelled data. This step can involve creating synthetic glucose data that mimics real-world spike patterns observed in the labeled datasets. This technique is used to augment the training data, enabling the wider training for machine learning model 414. For example synthetic glucose data may be generated to represent complicated or unique spike patterns such as intra-spike dips or intra-spike peaks.

In some embodiments, the generation of synthetic glucose spikes may be performed using a synthetic spike model implemented as a generative adversarial networks (GANs). The training model can be trained on labeled glucose data to generate synthetic examples that closely mimic real-world spikes and other trends, including features such as sharp rises, intra-spike dips, and gradual falls. These synthetic examples are added to the training dataset, enhancing the model's exposure to varied spike patterns. By augmenting the training data for machine learning model 414 with realistic synthetic spikes, machine learning model 414 is better equipped to handle edge cases and rare events, which improves its capability to distinguish between recognized and false events. This improvement in the diversity of training data directly enhances the model's accuracy in real-world applications, reducing the likelihood of undetected spikes and false alerts

Feature engineering techniques used include imputation (filling in missing values), discretization (converting continuous variables into discrete bins), categorical encoding (transforming categorical variables into numerical format), feature splitting (dividing a feature into multiple components), handling outliers (identifying and managing extreme values), variable transformations (applying mathematical transformations to variables), and scaling (normalizing the range of features). This ensures that the model is provided with informative and non-redundant data so that training is efficient. This ensures that the model is provided with informative and non-redundant data so that training is efficient.

After the training phase, the performance of machine learning model 414 may be evaluated using evaluation datasets, which may be datasets that were not used as part of the training. In some embodiments, an evaluation dataset may include actual datasets or synthetic datasets generated to evaluate model performance for specific spike patterns. The model's predictions are compared against the actual outcomes using various evaluation metrics. In particular, performance may be compared using metrics such as accuracy, precision, recall, F1-score, and confusion matrix.

Once the model achieves satisfactory performance, it is ready for deployment. Deployment refers to integrating the trained model into an application or system where it can make predictions on new, unseen data. The data input to the trained model generally mirrors the data used to train the model. In particular, the data input to the model once deployed typically includes features such as user demography, behavior metrics, transaction records, or sensor readings, depending on the application. This data should be preprocessed in the same way as the training data to ensure consistency. The output of the trained model is the predicted label or value, such as a classification result, a regression value, or a probability score, which should match the type of label used during training unless additional post-processing is applied. In some embodiments, the model may be exposed as an API, embedded into a software application, or used in batch processing pipelines. In some embodiments, the model is used in real-time decision-making systems, such as recommendation engines, fraud detection systems, or predictive maintenance applications. The deployed model may be monitored over time to ensure it continues to perform as expected and does not degrade due to changes in input data or system environments.

The training and evaluation of machine learning model 414 and subsequent deployment within machine learning architecture 410 improves the performance of glucose spike detection in real-time applications, such as a glucose monitoring application installed on user device 135. In one aspect, accuracy of glucose spike detection is improved resulting in fewer identification of false positives and negatives.

Machine learning system 400 may be used in various personal applications. As one non-limiting example, one application of the real-time spike detection functionality in machine learning system 400 is providing users with real-time feedback or coaching. Machine learning system 400 may utilize machine learning model 414 to monitor glucose levels (e.g., analyte data 404). When machine learning model 414 classifies a current measurement as being “in spike,” machine learning system 400 may further compute the area under the curve for the spike, which may still be ongoing and not ended at this point in time, and may assign the spike a point value based on the area under the curve. For example, the point value may be an integer between a range of values, such as between 0 and 100. Machine learning system 400 may display the point value to the user. The point value provides a quantitative measure of the spike. Machine learning system 400 (or a component in machine learning system 400) may continue these steps as additional glucose measurements become available, i.e., identifying whether the measurement belongs or does not belong to the same spike. If so, then machine learning system 400 (or a component in machine learning system 400) may recompute the area under the curve for the spike based on the current and past measurements that are associated with the same spike. And as before, machine learning system may convert the updated area under the curve value to a point system and displayed. In some embodiments, this process may iterate until machine learning model 414 detects that a glucose measurement does not belong to the same spike. In some embodiments, this determination may be confirmed by guardrail component 416. At that point, machine learning model 414 may conclude that the spike has ended and provide additional visualization for display to indicate the conclusion.

FIG. 4B is a block diagram of an exemplary machine learning system 400B for generating predicted wellness events based user analyte data. In some embodiments, one or more components of machine learning system 400 may be implemented in continuous analyte monitoring (CAM) system 100, according to some embodiments.

In some embodiments, input 402 may comprise user information 406, which can be provided from a number of sources including sources associated with the user such as analyte sensor 102, data receiving device 120, and user device 135 and data storage sources such as remote application server 155 or any other connected database. User information 406 may include but is not limited to meal information, level of physical activity, and physiological data about the user such as heartbeat, stress, and temperature. User information 406 may be provided as additional input to machine learning architecture 410 for generating predicted wellness events. In some embodiments, machine learning model 414 is trained to utilize user information 406 to improve the accuracy of predicted wellness devices based on real-time analyte data 404 alone.

In this manner, machine learning model 414 provides additional technical advantages over a conventional rules-based approach, which does not incorporate additional inputs such as user cohort information, meal information, and physical activity. Accordingly, user information 406 may include these additional inputs. In some embodiments, machine learning model 414 may be trained from a baseline model to a hybrid model for receiving multi-modal inputs such as user analyte data (current and/or historical), user cohort information (e.g., demographics), meal information, and physical activity.

In some embodiments, the baseline model may be initially trained based on learning patterns from user analyte current and historical data (labelled or unlabeled). Methods for implementing machine learning architecture 410 may be based on recurrent architectures for handling time-based sequential data and identifying patterns within this data. In some embodiments, tree-based models may be utilized as a baseline model, if the initial user analyte data lacks sufficient data. The baseline model that can detect previous wellness events—like sudden spikes, dips in the analyte data—and, in some embodiments, accurately predict imminent wellness events.

In some embodiments, the baseline model may then be further trained to receive as input additional data modalities that can further refine prediction performance for machine learning model 414. In addition to analyte readings, machine learning architecture 410 may incorporate physiological signals (e.g., heart rate, sleep quality from wearables), user behavioral logs (such as app usage, exercise patterns), and contextual data (like meal times, stress questionnaires, or environmental factors).

In some embodiments, the new inputs may be appended to the existing set of analyte-based inputs and fed into machine learning model 414. In some embodiments, machine learning model 414 may incorporate additional models trained for to handle different input streams where each separate model (or sub-model) may be dedicated to each data modality. For example, one model may receive as input continuous analyte signals, another model may receive as input user activity logs, and yet another model for meal information. The output of each model may then be merged within a final layer of machine learning model 414 for generating wellness event predictions.

In some embodiments, machine learning system 400 includes scheduler component 422 for scheduling delivery of analyte data 404 aggregated over a different predetermined time period to retrospective update component 418. In some embodiments, scheduler component 422 may schedule the delivery of analyte data 404 to retrospective update component 418 that is aggregated over a longer predetermined period of time than is received by the real-time prediction component 412 (e.g., one hour versus one minute, twelve hours versus five minutes, twenty hours versus five minutes). In some embodiments, scheduler component 422 may schedule delivery of analyte data 404 to retrospective update component 418 that is aggregated over predetermined period of time that overlaps the aggregated analyte data 404 that is received by real-time prediction component 412. For example, the scheduler component 422 may schedule delivery of analyte data 404 that is from time t₋₂₀ to t₊₂₀ while the real-time prediction component 412 receives analyte data 404 that is from t₋₁₅ to t₊₅.

In some embodiments, retrospective update component 418 runs independently of any output of real-time prediction component 418 and identifies missed predicted wellness events based only on analyte data 404 that is received from scheduler component 422. Examples of missed predicted wellness events include false negatives or false positives discussed above. In such embodiments, graphical user interface 420 may be configured to combine the predicted wellness event sequence (and any modifications provided by guardrail component 416, if applicable) from real-time prediction component 412 and any missed predicted wellness events from retrospective update component 418 to provide a comprehensive display of predicted wellness events in a time-ordered sequence for a predetermined time period. Graphical user interface 420 may continuously receive predicted wellness event sequences and missed predicted wellness events from machine learning architecture 410 and update a display each time new event sequence and missed predicted wellness events are received. That is, the predetermined time period that graphical user interface 420 may continuously display predicted wellness event sequences is different from the time period of the aggregated analyte data 404 provided by analyte sensor 102. For example, graphical user interface 420 may continuously update a display each time a predicted wellness event sequence for a particular time period (e.g., every five minutes) and any missed predicted wellness events for that same time period is received from machine learning architecture 410. The display provided by graphical user interface 420 may span a longer time window, such as a twelve hour time period, and this display may be updated in increments based on the time period of each predicted wellness event sequence. Predicted wellness event sequences are discussed in further detail in FIG. 5.

In some embodiments, retrospective update component 418 runs in conjunction with output from real-time prediction component 412 and identifies missed predicted wellness events based on both analyte data 404 (from scheduler component 422) and on any output provided by real-time prediction component 412. That is, output from real-time prediction component 412 may be provided to retrospective update component 418, and in combination with analyte data 404 provided via scheduler component 422, retrospective update component 418 may identify and output missed wellness event sequences that were not identified by real-time prediction component 412 within that time period.

### Exemplary Wellness Event Sequence

FIG. 5 is an exemplary predicted wellness event sequence 500, according to some embodiments. Predicted wellness event sequence 500 may comprise predicted wellness events and associated event data surrounding the predicted wellness events, both of which may be presented in a time-ordered visualization. Predicted wellness event sequence 500 may display predicted wellness events, such as one or more predicted glucose crashes or one or more predicted glucose spikes, and other event data associated with the predicted wellness events, such as glucose data between (i.e., preceding and succeeding) the predicted wellness events. For example, predicted wellness event sequence 500 depicts events and event data over a predetermined period of time (e.g., twelve hours, twenty four hours) on the y-axis and analyte amount (e.g., mg/dl) on the x-axis.

A non-limiting example of a predicted wellness event is a glucose spike 504 which may include event indications such as a spike start 502 at time t₁, area under the spike curve 506, and a spike end 508 at time t₂. Predicted wellness event sequence 500 may additionally display event data (e.g., glucose levels) preceding spike start 502 at time t₁ and after spike end 508 at time t₂. In some embodiments, the event data comprises analyte data 404 from analyte sensor 102 and the event (glucose spike 504) and event indications (spike start 502, spike end 508) are predicted by machine learning architecture 410 and provided for display with the analyte data 404 by graphical user interface 420. It is understood that predicted wellness event sequence may comprise one or more glucose spikes and that glucose spike 504 is merely exemplary. Additionally, graphical user interface 420 may be configured to display one or more of the predicted wellness events and event indications; for example, graphical user interface 420 may be configured display predicted wellness events but not the area under the curve information.

In some embodiments the spike times, including both the spike start 502 and spike end 508, are non-overlapping within the predicted wellness event sequence. Non-overlapping may mean discrete times within the time span represented by the predicted wellness event sequence.

Another non-limiting example of a predicted wellness event is a glucose crash 512 which may include event indications such as a crash start 510 at time t₃, area under the crash curve 514, and a crash end 516 at time t₄. Similar to glucose spike 504, predicted wellness event sequence 500 may additionally display additional event data (e.g., glucose levels) preceding crash start 510 at time t₃ and after crash end 516 at time t₄. In some embodiments, the event data comprises analyte data 404 from analyte sensor 102 and the event (glucose crash 512) and event indications (crash start 502, crash end 508) are predicted by machine learning architecture 410 and provided for display with the analyte data 404 by graphical user interface 420.

### ML Architecture Operation

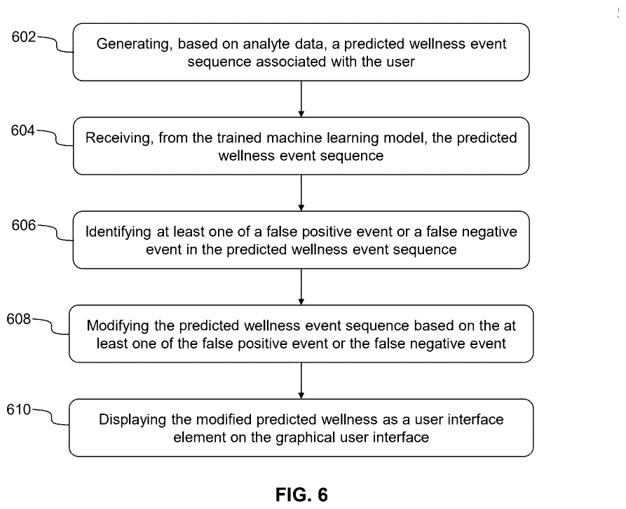

FIG. 6 is a flowchart illustrating a method 600 for utilizing a continuous analyte monitoring system within a home environment, according to some embodiments. Method 600 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-4, one or more functions described with respect to FIG. 6 may be performed by a components of CAM system 100 and/or machine learning system 400, either separately or in a distributed fashion. In such an embodiment, any of these components may execute code in memory to perform certain steps of method 600 of FIG. 6. While method 600 of FIG. 6 will be discussed below as being performed by certain components of machine learning system 400, other components may store the code and therefore may execute method 600 by directly executing the code. Accordingly, the following discussion of method 600 will refer to components of FIGS. 1-4 as an exemplary non-limiting embodiment. Moreover, it is to be appreciated that not all steps may be needed to perform the disclosure provided herein. Further, some of the functions may be performed simultaneously, in a different order, or by the same components than shown in FIG. 6, as will be understood by a person of ordinary skill in the art.

In 602, machine learning model 414 generates a predicted wellness event sequence based on analyte data 404 received from the in vivo analyte sensor 102 associated with a user. Machine learning model 414 may be trained to receive, as input, analyte data 404 for a predetermined period of time or in periodic increments (e.g., every one minute, every two minutes) and generate predicted wellness events based on the received analyte data.

In some embodiments, the predetermined period of time that machine learning model 414 receives analyte data 404 is configurable based on one or more of the needs of the user, the capabilities of the device on which machine learning model 414 is installed, any improvements in the training of machine learning model 414, and any imposed accuracy and display requirements (e.g., from a regulatory agency). Increasing the predetermined period of time may increase accuracy of the predicted wellness events but at the cost of providing real-time information to graphical user interface 420. Decreasing the predetermined period of time may decrease accuracy of the predicted wellness events but at the benefit of providing information more quickly to graphical user interface 420.

In some embodiments, machine learning model 414 is implemented as part of a machine learning architecture, such as machine learning architecture 410, and may be implemented as software running on a user device, such as a smartphone or tablet. Machine learning model 414 may be trained to identify, or predict, certain wellness events such as glucose spikes and glucose crashes as well as related event data such as start and end points for glucose spikes and glucose crashes.

In some embodiments, output of the machine learning model 414 includes the predicted wellness events based on the provided analyte data 404 for that incremental time period. In some embodiments, output of the machine learning model 414 is a predicted wellness event sequence that comprises the predicted wellness events organized in time-order based on the timing information included in analyte data 404.

In 604, guardrail component 416 is configured to receive the predicted wellness events or predicted wellness event sequence from machine learning model 414.

In 606, Guardrail component 416 is configured to identify any false positives or negatives in the received wellness events or predicted wellness event sequence. In some embodiments, guardrail component 416 identifies false negative events and false positive events by performing an analysis of one or more sub-sequence of time-ordered predicted wellness events, identifying any patterns within the sub-sequence, and determining whether any predicted wellness events deviates from the identified patterns. In this manner, the predicted wellness event sequence provided by machine learning model 414 may be considered an initial prediction based on analyte data 404.

As one non-limiting example, predicted wellness event sequence may be time-ordered based on analyte data 404. The predicted wellness event sequence may include a sub-sequence comprising predicted glucose spikes or glucose crashes for a time-ordered sequence. Guardrail component 416 may be configured to identify a pattern within that subsequence of predicted glucose spikes or crashes and subsequently identify any deviations from that pattern (such as a missing spike or crash within a pattern of spikes and crashes). Guardrail component 416 may then be configured to fix the predicted wellness event sequence by adding corrected wellness events to the sequence, such as by correcting a false glucose spike and a false glucose crash.

In 608, guardrail component 416 modifies, or corrects, the predicted wellness event sequence based on the at least one of the false positive event or the false negative event. In some embodiments, guardrail component 416 may be configured to perform these corrections by muting or continuing the wellness event. For example, if guardrail component 416 identifies a false glucose spike, guardrail component 416 may be configured to mute the spike thereby correcting it from being identified as glucose spike within the predicted wellness event sequence.

In some embodiments, guardrail component 416 may be configured to identify certain spikes that are a result of a series of false negatives that occur either at the beginning (e.g., spike start 502) or at the end of a spike (e.g., spike end 508). Guardrail component 416 may identify this pattern of false negatives as being different from those that arise in the middle of a spike (e.g., glucose spike 504), which may appear as drops in spike.

In some embodiments, guardrail component 416 is implemented as part of machine learning model 414 and not as a separate component. In some embodiments, step 604 is optional because output from machine learning model 414 may be considered to be sufficiently accurate (e.g., confidence in the predicted wellness events meets a configurable confidence threshold) so that additional processing for false negatives and/or false positives by guardrail component 416 is not necessary.

In 610, real-time prediction component 412 provides the predicted wellness event sequence with any corrected false negatives and false positives, or a modified predicted wellness event, to graphical user interface 420 for display, as a user interface element, such as those shown in FIG. 5. User interface elements are modified to correspond to predicted wellness events and event data included in the predicted wellness event sequence, such as glucose spikes and glucose crashes.

In some embodiments, where user information 406 is provided to machine learning architecture 410, graphical user interface 420 may also display user information 406 as user interface elements in relation to the corresponding event data provided in predicted wellness event sequence. For example, user information 406 may include other events not tied to analyte data 404 such as meals, exercise, and sleeping. These events may be displayed as user interface elements on graphical user interface 420 in time-ordered sequence with the predicted wellness events and event data. For example, if user input 402 indicates that a user had consumed a meal around time t₁, as indicated in FIG. 5, graphical user interface 420 may display a user interface element in proximity to spike start 502 to indicate the occurrence of the meal at t₁. The juxtaposition of predicted wellness events and user information related elements enables graphical user interface 420 to provide additional information to a user.

### Missed Predicted Wellness Events

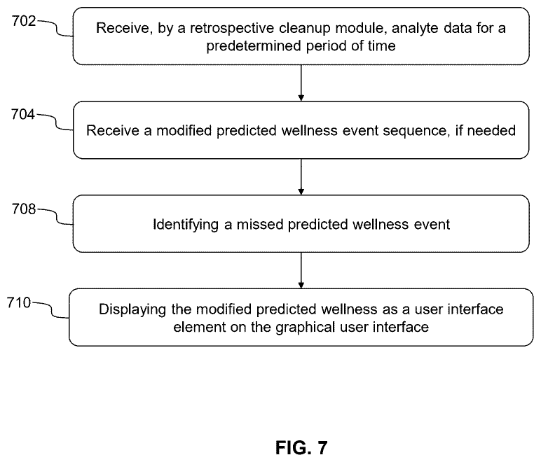

FIG. 7 is a flowchart illustrating a method 700 for utilizing detecting missed predicted wellness events in a predicted wellness event sequence, according to some embodiments. Method 700 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-4, one or more functions described with respect to FIG. 7 may be performed by a components of CAM system 100 and/or machine learning system 400, either separately or in a distributed fashion. In such an embodiment, any of these components may execute code in memory to perform certain steps of method 700 of FIG. 7. While method 700 of FIG. 7 will be discussed below as being performed by certain components of machine learning system 400, other components may store the code and therefore may execute method 700 by directly executing the code. Accordingly, the following discussion of method 700 will refer to components of FIGS. 1-4 as an exemplary non-limiting embodiment. Moreover, it is to be appreciated that not all steps may be needed to perform the disclosure provided herein. Further, some of the functions may be performed simultaneously, in a different order, or by the same components than shown in FIG. 7, as will be understood by a person of ordinary skill in the art.

In 702, retrospective update component 418 receives analyte data 404 for a predetermined period of time (i.e., in specified time increments). As noted above, time increments of analyte data 404 received by retrospective update component 418 is different from the time increments of analyte data 404 that is received by real-time prediction component 412. Retrospective update component 418 is configured to identify any missed prediction wellness events from analyte data 404. The accuracy of detecting missed prediction wellness events is enhanced by receiving more analyte data 404 for a larger predetermined period of time. The predetermined period of time that retrospective cleanup component may be configurable based on the needs of the user, the capabilities of the device on which retrospective update component 418 is installed, any improvements in the training of retrospective update component 418, and any imposed accuracy and display requirements (e.g., from a regulatory agency).

In 704, in some embodiments, retrospective update component 418 receives a modified predicted wellness event sequence from real-time prediction component 412. In some embodiments, retrospective update component 418 operates independently of real-time prediction component 412 and functions based only analyte data 404. Whether retrospective update component 418 receives the modified predicted wellness event sequence is configurable.

In 706, retrospective update component 418 identifies any missed predicted wellness event based on the analyte data 404. In embodiments where retrospective update component 418 does not receive the output from real-time prediction component 412 (i.e., is independent from real-time prediction component 412), retrospective update component 418 generates the predicted wellness events based on the received analyte data 404 and graphical user interface 420 performs a compare and merge operation on the output from both the real-time prediction component 412 and retrospective update component 418. This operation identifies any missed predicted wellness events in the output of the real-time prediction component 412. Graphical user interface 420 may then update the predicted wellness event sequence to include any missed predicted wellness events that were identified by retrospective update component 418.

In embodiments where retrospective update component 418 does receive output from real-time prediction component 412, retrospective update component 418 may both the predicted wellness events based on the received analyte data 404 and perform the update operation on the predicted wellness event sequence to include any missed wellness events that were not identified by real-time prediction component 412.

In 708, graphical user interface 420 receives and displays the output from retrospective update component 418 as a predicted wellness event sequence, such as shown in FIG. 5.

### Model Training Architecture

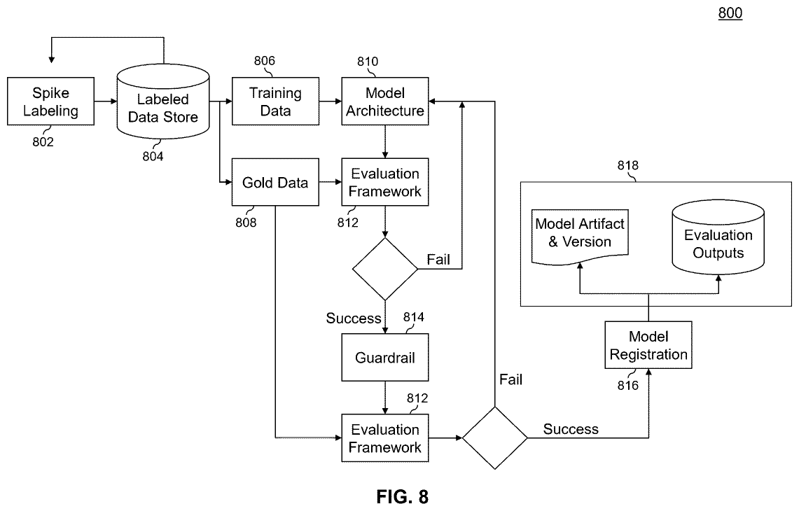

FIG. 8 is a flow diagram illustrating a components of a model training architecture 800 and communications between the components, according to some embodiments.

Model training architecture 800 includes a labeled data store 804, model architecture 810, evaluation framework 812, guardrail component 814, model registration 816, and model storage 818. In some embodiments, model training architecture 800 may be used for both initial training of a machine learning model (e.g., machine learning model 414) and retraining of the machine learning model.

Labeled data store 804 receives spike labeling data 802, which includes training samples that may have ground truth labels indicating whether data are events (e.g., spike) or event indications (e.g., spike start, spike end, spike peak). Spike labeling data 802 may include both real-world data or synthetic data, which can be helpful to generate synthetic examples that represent unusual, rare, or new trends or patterns in data to enhance the model's exposure to new, different, and rare spike patterns.

Spike labeling data 802 may include glucose time series data annotated based of clinician rules. In some embodiment, annotations are based on specific rules and standards that clinicians use to identify spikes. Spike labeling data 802 may be retrieved from multiple (e.g., hundreds) of glucose sensors to ensure variety of data from multiple input sources.

Labeled data store 804 uses the spike labeling data 802 as training data 806 to model architecture 810 and as gold data 808 to evaluation framework 812 for evaluating the output of model architecture 810 and guardrail component 814. Gold data 808 includes data that is used for verifying the output of model architecture 810 for the purpose of determining the assessing model performance.

Model architecture 810 may be trained using training data 806 that can include training samples of glucose values (e.g., a current value being classified, 18 previous values, and 5 subsequent values). During each training iteration, model architecture 810 may process and generate predictions based on training data 806. These predictions are provided to evaluation framework 812 for comparing predictions to ground truth labels of the training sample according to a loss function and, based on the result of the comparison, iteratively update model architecture 810. Evaluation framework 812 may determine based on threshold success conditions when model architecture 810 has successfully been trained. Training may continue iteratively until evaluation framework determines that a terminating condition is met. For example, training could continue until the differences between the model's predictions and the ground truth labels are within a threshold range. As another example, training could terminate after a sufficiently large number of training iterations have been completed.

Upon meeting the success condition, guardrail component 814 is trained next using the output from model architecture 810. Guardrail component 814 may be configured with rules to process the output to identify false positives or false negatives within the predictions of model architecture 810. Evaluation framework 812 performs a similar iterative evaluation and training of guardrail component 814 based on threshold success conditions. If the success condition is not met, evaluation framework 812 may return the training back to model architecture 810 for continuing the training process.

Upon meeting the success condition for guardrail component 814, evaluation framework 812 may register the trained model architecture 810 and guardrail component 814 in a database, model registration 816. Results of the evaluation and information of the model architecture 810 and guardrail component 814 may be further archived in model storage 818.

In some embodiments, machine learning models may be retrained using model training architecture 800, which may be implemented to monitor machine learning models to identify model drift within the models. Model training architecture 800 can be configured to continuously track model performance and detect any deviations from expected behavior. For example, evaluation framework 812 may be configured with monitoring capabilities to monitor outputs from machine learning models after they have been trained. When evaluation framework 812 identifies model drift in a machine learning model, it can trigger the retraining cadence and prompts a reassessment of the machine learning model. This approach ensures that model output remains accurate and reliable over time, adapting to new data and evolving patterns in spike data.

The machine learning model can be retrained on a regular schedule to ensure it remains accurate and up-to-date. The cadence for retraining can be dynamic based on the specific requirements and the rate at which new glucose data is collected. Examples of retraining intervals include monthly (e.g., for environments where data is rapidly evolving), quarterly (for more stable environments with moderate data changes), biannually or annually (e.g., for very stable environments with minimal changes to data). Overall, this cadence approach enables machine learning architecture 800 to ensure that models deliver consistent and reliable outcomes, enhancing the trust and effectiveness of the solutions provided.

When retraining is triggered, model training architecture 800 may be configured to perform retraining steps including data collection, data preprocessing, and model training.

When performing data collection, model training architecture 800 may receive new glucose time series data that is continuously collected from glucose sensors and also may include synthetically generated glucose data. This data can include both previously unseen patterns and any new annotations made by clinicians. The glucose sensors may be associated with various users and the glucose data provided by each sensor may be anonymized before being provided to model training architecture 800. The new glucose time series data may be undergo an annotation process that includes annotating events (e.g., spikes) and event indications (e.g., spike start, spike end) according to established rules. This ensures that the training data that model training architecture 800 uses to retrain machine learning models remains consistent and accurate.

Model training architecture 800 may further perform data preprocessing on the annotated data to clean and normalize to ensure the data is suitable for training. This step may include handling missing values, smoothing, and scaling of data. After these steps, the new data is prepared for retraining the model.

For retraining the model based, model training architecture 800 performs steps of splitting the updated data set, which includes dividing the data into training, validation, and test sets. Training data 806 may be updated to include the new training data and is provided for training model architecture 810. Evaluation framework 812 may then validate output from model architecture 810 which evaluates the model based on the validation dataset. This step may include tuning hyperparameters and preventing overfitting. Evaluation framework 812 may then assess the model performance based on the test set to ensure accuracy of the output provided by model architecture 810. This assessment also includes evaluating performance against key performance metrics, such as accuracy, precision, recall, and F1 score.

Evaluation framework 812 may then be configured to deploy the machine learning model into the production environment (i.e., into model registration 816) after successful validation, the updated model is deployed into the production environment.

In some embodiments, machine learning architecture 800 may include a connection to clinician devices as part of a feedback loop for receiving input from clinicians regarding real-world outputs of machine learning models. The input may indicate whether predicted wellness events were correct and the input may be used to further refine and improve the model in subsequent retraining cycles.

### CONCLUSION

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A system for generating a graphical user interface comprising user interface elements representative of a subject's wellness based at least in part on analyte data received from an in vivo analyte sensor, wherein the analyte data represents data collected from the in vivo analyte sensor over a predetermined period of time, the system comprising:
a trained machine learning model configured to generate, based on the analyte data received from the in vivo analyte sensor, a predicted wellness event sequence associated with the subject;
a guardrail component configured to:
receive, from the trained machine learning model, the predicted wellness event sequence;
identify, in the predicted wellness event sequence based on an analysis of time-ordered events within the predicted wellness event sequence, at least one of a false positive event or a false negative event in the predicted wellness event sequence; and
modify the predicted wellness event sequence based on the at least one of the false positive event or the false negative event;
a display configured to display, as a user interface element on the graphical user interface, the modified predicted wellness event sequence.

2. The system of claim 1, wherein the predicted wellness event sequence comprises one or more predicted glucose spikes within the predetermined period of time.

3. The system of claim 2, wherein the predicted wellness event sequence comprises spike data including one or more predicted spike start times and one or more predicted spike end times within the predetermined period of time.

4. The system of claim 1, wherein the predicted wellness event sequence comprises one or more predicted glucose crashes within the predetermined period of time.

5. The system of claim 4, wherein the predicted wellness event sequence comprises crash data including one or more predicted crash start times and one or more predicted crash spike end times.

6. The system of claim 1, wherein the false positive event comprises a false glucose spike or a false glucose crash and the false negative event comprise a missed glucose spike or a missed glucose crash.

7. The system of claim 6, wherein the analysis perform of the time-ordered events comprises at least one of:
identify a false glucose spike predicted by the trained machine learning model;
identify a false glucose crash predicted by the trained machine learning model;
identify the missed glucose spike not predicted by the trained machine learning model; or
identify the missed glucose crash not predicted by the trained machine learning model.

8. The system of claim 1, the system further comprising:
a retrospective update module configured to identify, based on the analyte data, a missed predicted wellness event in the modified predicted wellness sequence; and
wherein the display is further configured to display, as a second user interface element on the graphical user interface element, the missed predicted wellness event, and
wherein the missed predicted wellness event is displayed concurrently with the modified predicted wellness sequence.

9. The system of claim 1, wherein the predetermined period time comprises a 5 minute interval.

10. The system of claim 1, wherein the predetermined period of time comprises a 1 minute interval.

11. The system of claim 1, wherein the predetermined period of time comprises a 24 hour interval.

12. The system of claim 1, wherein the system is implemented as a mobile device.

13. A method generating a graphical user interface comprising user interface elements representative of a subject's wellness based at least in part on analyte data received from an in vivo analyte sensor, wherein the analyte data represents data collected from the in vivo analyte sensor over a predetermined period of time, the method comprising:
generating, by a trained machine learning model, based on a portion of the analyte data received from the in vivo analyte sensor, a predicted wellness event sequence associated with the subject;
receiving, from the trained machine learning model, the predicted wellness event sequence;
identifying, in the predicted wellness event sequence based on an analysis of time-ordered events within the predicted wellness event sequence, at least one of a false positive event or a false negative event in the predicted wellness event sequence; and
modifying the predicted wellness event sequence based on the at least one of the false positive event or the false negative event;
displaying, as a user interface element on the graphical user interface, the modified predicted wellness event sequence.

14. The method of claim 13, wherein the predicted wellness event sequence comprises one or more predicted glucose spikes within the predetermined period of time.

15. The method of claim 14, wherein the predicted wellness event sequence comprises spike data including one or more predicted spike start times and one or more predicted spike end times within the predetermined period of time.

16. The method of claim 13, wherein the predicted wellness event sequence comprises one or more predicted glucose crashes within the predetermined period of time.

17. The method of claim 16, wherein the predicted wellness event sequence comprises crash data including one or more predicted crash start times and one or more predicted crash spike end times.

18. The method of claim 13, wherein the false positive event comprises a false glucose spike or a false glucose crash and the false negative event comprise a missed glucose spike or a missed glucose crash.

19. The method of claim 18, wherein the analysis perform of the time-ordered events comprises at least one of:
identifying a false glucose spike predicted by the trained machine learning model;
identifying a false glucose crash predicted by the trained machine learning model;
identifying the missed glucose spike not predicted by the trained machine learning model; or
identifying the missed glucose crash not predicted by the trained machine learning model.

20. The method of claim 13, wherein the analyte data comprises a second portion of analyte data for a second period of time, the method further comprising:
identifying, based on the analyte data, a missed predicted wellness event in the modified predicted wellness sequence; and
displaying, as a second user interface element on the graphical user interface element, the missed predicted wellness event, wherein the missed predicted wellness event is displayed concurrently with the modified predicted wellness sequence.
